(19) United States
(12) Patent Application Publication
(10) Pub. No.: US 2024/0009344 A1
(43) Pub. Date: Jan. 11, 2024

(54) ABSORBENT BONE HEMOSTATIC MATERIAL COMPOSITION COMPRISING ANTIBIOTIC AND PREPARATION METHOD THEREFOR

(71) Applicant: THERACION BIOMEDICAL CO. LTD., Seongnam-si, Gyeonggi-do (KR)

(72) Inventors: Eun Jin KIM, Seongnam-si (KR); Byung Nam KIM, Chungcheongbuk-do (KR)

(73) Assignee: THERACION BIOMEDICAL CO. LTD., Seongnam-si, Gyeonggi-do (KR)

(21) Appl. No.: 18/039,551

(22) PCT Filed: Oct. 5, 2021
(86) PCT No.: PCT/KR2021/013596

§ 371 Date: May 31, 2023

(30) Foreign Application Priority Data
Dec. 2, 2020 (KR) 10-2020-0166202

(51) Int. Cl. A61L 24/04; A61L 24/00

(52) U.S. Cl. CPC A61L 24/043; A61L 24/0015; A61L 24/0042; A61L 2400/04; A61L 2300/406; A61L 2300/232

(57) ABSTRACT

An absorbent bone hemostatic material composition and a method for preparing the composition are disclosed. The composition includes an antibiotic and absorbent bone hemostatic materials that provide a physical protective film for stopping bone hemorrhage during surgery. Thus, the composition containing the absorbent bone hemostatic materials and antibiotic exhibits an immediate hemostatic effect and prevent infection and inflammatory response at the surgical site.

### TECHNICAL FIELD

The present invention relates to an absorbent bone hemostatic material composition including an antibiotic and a preparation method therefor, and more specifically, to an absorbent bone hemostatic material that provides a physical protective film for stopping bone hemorrhage during surgery, thereby exhibiting an immediate hemostatic effect and preventing infection and inflammatory response at the surgical site.

### BACKGROUND ART

Bleeding is one of the obstacles that make surgery difficult, and it is very important to stabilize bleeding because a lot of blood loss may affect normal body functions. Hemostasis requires the use of blood coagulants or vascular occlusion devices such as gauze or hemostatic clamps. This type of hemostatic material utilizes the property of slowly activating coagulation factors or narrowing severed blood vessels. Numerous hemostatic materials have already been developed for application of soft tissue bleeding, but there are limitations in that the hemostatic materials cannot be applied to bone bleeding. This is because the blood flow during bone bleeding prevents the stable formation of blood clots, and the bone tissue itself maintains a structure through this condition. In the past, there have been many hemostatic materials that include non-biocompatible materials and thus remain in the body without being completely biodegraded. As a result of many studies, it has been confirmed that the components of the bone hemostatic materials have a negative effect on bone regeneration when the components of the bone hemostatic materials remain unbiodegraded.

Korean Patent Publication No. 10-2014-0107429 discloses a hemostatic agent and a method of using the same. The hemostatic agent includes reverse micelles having an outer hydrophobic shell of suitable biocompatible hydrophobic components such as alkanes, and hydrophilic positively charged chitosan moieties enclosed within the hydrophobic shell, thereby attenuating or stopping the bleeding. However, there are limitations in that the application to bone hemostasis and the immediate hemostasis by a physical protective film are not disclosed.

Korean Patent Publication No. 10-2018-0055747 discloses a novel hemostatic agent including a mussel adhesive protein and a method for preparing an absorbent bone hemostatic agent by using the same. The application to bone hemostasis is not disclosed. Since a lot of auxiliary materials such as nanofiber hemostatic dressings, hemostatic sponges and patches are required and it is difficult to biodegrade well in vivo, it is highly likely to interfere with bone formation. Therefore, there is a need to develop a bone hemostatic material that is quickly absorbed and biodegraded so as not to interfere with bone formation and has an immediate hemostatic effect.

Accordingly, the inventors of the present application have studied to solve the above problems and found that a bone hemostatic material composition including a specific polymer compound and an antibiotic had an immediate hemostatic effect, had a minimal effect on bone regeneration, and could also prevent infection and inflammatory response at the surgical site. In this manner, the inventors of the present application completed the present invention.

### DESCRIPTION OF EMBODIMENTS

### Technical Problem

The present invention has been made in an effort to solve the problems of the related art and aims to provide an absorbent bone hemostatic material composition including an antibiotic.

In addition, the present invention aims to provide an absorbent bone hemostatic material including the absorbent bone hemostatic material composition.

In addition, the present invention aims to provide a method for preparing the absorbent bone hemostatic material composition.

### Solution to Problem

As a technical means for achieving the above technical objectives, an aspect of the present invention provides an absorbent bone hemostatic material composition.

The absorbent bone hemostatic material composition includes: a first polymer including a compound represented by Formula 1 or a salt thereof as an active ingredient; a second polymer including a compound represented by Formula 2 or a salt thereof as an active ingredient; and an antibiotic.

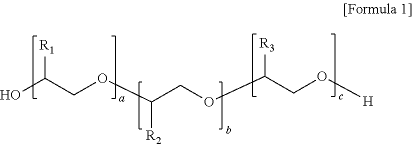

In Formula 1, R1 to R3 are each independently hydrogen, linear or branched C1-C10 alkyl, linear or branched C1-C10 alkoxy, linear or branched C1-C10 aminoalkyl, linear or branched C2-C10 alkenyl, or linear or branched C2-C10 alkynyl, a and c are each independently an integer of 2 to 128, and b is an integer of 16 to 67.

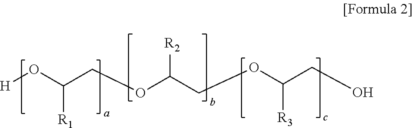

In Formula 2, R1 to R3 are each independently hydrogen, linear or branched C1-C10 alkyl, linear or branched C1-C10 alkoxy, linear or branched C1-C10 aminoalkyl, linear or branched C2-C10 alkenyl, or linear or branched C2-C10 alkynyl, a and c are each independently an integer of 2 to 128, and b is an integer of 16 to 67.

In Formulae 1 and 2, R1 to R3 may each independently be hydrogen, linear or branched C1-C5 alkyl, linear or branched C2-C5 alkenyl, or linear or branched C2-C5 alkynyl.

In Formulae 1 and 2, R1 to R3 may each independently be hydrogen or linear or branched C1-C5 alkyl.

In Formulae 1 to 2, R1 and R3 may each be hydrogen, and R2 may be linear or branched C1-C3 alkyl.

The antibiotic may include a material selected from the group consisting of first-generation cephapirin, second-generation ceftazidime, third-generation cefotaxime, and fourth-generation cefepime in cephalosporins, piperacillin in penicillins, gentamicin, amikacin, tobramycin, and neomycin in aminoglycosides, first-generation norfloxacin, second-generation ciprofloxacin, third-generation levofloxacin, and fourth-generation trovafloxacin in quinolones, tetracycline in tetracyclines, ertapenem, meropenem, and imipenem/cilastatin in carbapenems, vancomycin in glycopeptides, complexes thereof, derivatives thereof, and combinations thereof.

50-70 parts by weight of the second polymer and 0.1-20 parts by weight of the antibiotic may be included based on 30-50 parts by weight of the first polymer.

A weight average molecular weight of the first polymer may be 7,000 to 10,000.

A weight average molecular weight of the second polymer may be 6,000 to 12,000.

The absorbent bone hemostatic material composition may be biodegraded by 90% or more after 3 hours in a phosphate buffer solution (PBS) of pH 7 to 8 at a temperature of 30° C. to 37° C.

The absorbent bone hemostatic material composition may exhibit an immediate hemostatic ability of 0-10 seconds.

The absorbent bone hemostatic material composition may have a bacteriostatic reduction rate of 99% or more after 7 days against Staphylococcus aureus or Escherichia coli.

In addition, another aspect of the present invention provides an absorbent bone hemostatic material.

The absorbent bone hemostatic material includes the absorbent bone hemostatic material composition.

The absorbent bone hemostatic material may be at least one formulation selected from semi-solid formulation and solid formulation.

In addition, another aspect of the present invention provides a method for preparing an absorbent bone hemostatic material composition.

The method for preparing the absorbent bone hemostatic material composition includes: adding, to a reactor, a first polymer including a compound represented by Formula 1 or a salt thereof as an active ingredient, a second polymer including a compound represented by Formula 2 or a salt thereof as an active ingredient, and an antibiotic; heating and stirring the mixture added to the reactor at a temperature of 50° C. to 100° C.; and cooling the mixture.

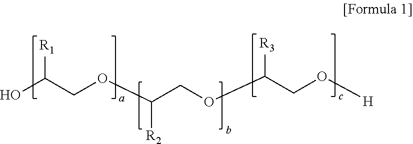

In Formula 1, R1 to R3 are each independently hydrogen, linear or branched C1-C10 alkyl, linear or branched C1-C10 alkoxy, linear or branched C1-C10 aminoalkyl, linear or branched C2-C10 alkenyl, or linear or branched C2-C10 alkynyl, a and c are each independently an integer of 2 to 128, and b is an integer of 16 to 67.

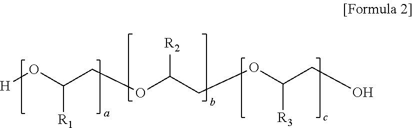

In Formula 2, R1 to R3 are each independently hydrogen, linear or branched C1-C10 alkyl, linear or branched C1-C10 alkoxy, linear or branched C1-C10 aminoalkyl, linear or branched C2-C10 alkenyl, or linear or branched C2-C10 alkynyl, a and c are each independently an integer of 2 to 128, and b is an integer of 16 to 67.

### Advantageous Effects of Disclosure

As described above, the absorbent bone hemostatic material composition according to the present invention provides an effect of being quickly absorbed and biodegraded in vivo.

The absorbent bone hemostatic material composition according to the present invention provides an immediate hemostatic effect.

The absorbent bone hemostatic material composition according to the present invention has an effect of being easily molded into a desired shape because the hardness of the absorbent bone hemostatic material composition varies with temperature. Accordingly, the absorbent bone hemostatic material composition according to the present invention provides an effect of increasing adhesive force.

The absorbent bone hemostatic material composition according to the present invention includes minimal materials, is harmless to the human body, and mixes properly so that the finished product has the effect of the raw material as it is.

The absorbent bone hemostatic material composition according to the present invention provides the effect of capable of preventing infection and inflammatory response at the surgical site.

### BRIEF DESCRIPTION OF DRAWINGS

FIG. 1 is a flowchart schematically illustrating a method for preparing an absorbent bone hemostatic material composition according to an embodiment of the present invention.

FIG. 2 is a graph showing the biodegradability of the absorbent bone hemostatic material composition according to an embodiment of the present invention.

FIG. 3 is a graph showing the compressive strength of bone hemostatic material compositions according to Examples of the present invention and Comparative Example.

FIG. 4 is a graph showing the adhesive strength of bone hemostatic material compositions according to Examples of the present invention and Comparative Example.

FIG. 5A to 5C are graphs showing the drug release ability of absorbent bone hemostatic material compositions according to Examples of the present invention.

FIG. 6 is a graph showing the hemostasis evaluation results of the absorbent bone hemostatic material composition over time according to an embodiment of the present invention.

FIGS. 7A and 7B are photographs showing the biodegradability of the bone hemostatic material composition according to an embodiment of the present invention in in vivo animal tests, respectively.

FIGS. 8A and 8B are graphs showing the antibacterial effect of the bone hemostatic material compositions over time according to Example of the present invention and Comparative Example, respectively, and FIGS. 8C and 8D are photographs showing the antibacterial effect of the bone hemostatic material compositions over time according to Example of the present invention and Comparative Example, respectively.

FIGS. 9A to 9D are photographs showing the inflammatory response and bone regeneration effect of the bone hemostatic material compositions over time according to Examples of the present invention and Comparative Examples.

### BEST MODE FOR CARRYING OUT THE INVENTION

Hereinafter, examples of the present invention will be described so that those of ordinary skill in the art may easily carry out the present invention. However, the present disclosure may be implemented in various different forms and is not limited to the embodiments described herein.

### Example 1. Preparation of Absorbent Bone Hemostatic Material Composition (Including 120 mg/g of Antibiotic)

In order to prepare the absorbent bone hemostatic material composition according to the present invention, 34.8 wt % of poloxamer 188, 54.5 wt % of polyethylene glycol-ran-propylene glycol, and 10.7 wt % of gentamicin sulfate were added to a reactor. Thereafter, the mixture introduced into the reactor was melted at a temperature of 80° C. and stirred for a sufficient time. Thereafter, the melted and stirred mixture was poured into a mold, placed in a cooler, and cooled at a temperature of 4° C. for 30 minutes to prepare a solidified absorbent bone hemostatic material composition (TWG-120).

### Example 2. Preparation of Absorbent Bone Hemostatic Material Composition (Including 60 mg/g of Antibiotic)

A solidified absorbent bone hemostatic material composition (TWG-60) was prepared in the same manner as in Example 1, except that 36.8 wt % of poloxamer 188, 57.5 wt % of polyethylene glycol-ran-propylene glycol, and 5.7 wt % of gentamicin sulfate were added to the reactor.

### Example 3. Preparation of Absorbent Bone Hemostatic Material Composition (Including 30 mg/g of Antibiotic)

A solidified absorbent bone hemostatic material composition (TWG-30) was prepared in the same manner as in Example 1, except that 37.9 wt % of poloxamer 188, 59.2 wt % of polyethylene glycol-ran-propylene glycol, and 2.9 wt % of gentamicin sulfate were added to the reactor.

### Comparative Example 1. Preparation of Absorbent Bone Hemostatic Material Composition (Including No Antibiotic)

A solidified absorbent bone hemostatic material composition (Tableau Wax) was prepared in the same manner as in Example 1, except that 39 wt % of poloxamer 188 and 61 wt % of polyethylene glycol-ran-propylene glycol were added to the reactor and no antibiotic was added thereto.

### Experimental Example 1. Biodegradability Measurement Experiment (Dissolution Test)

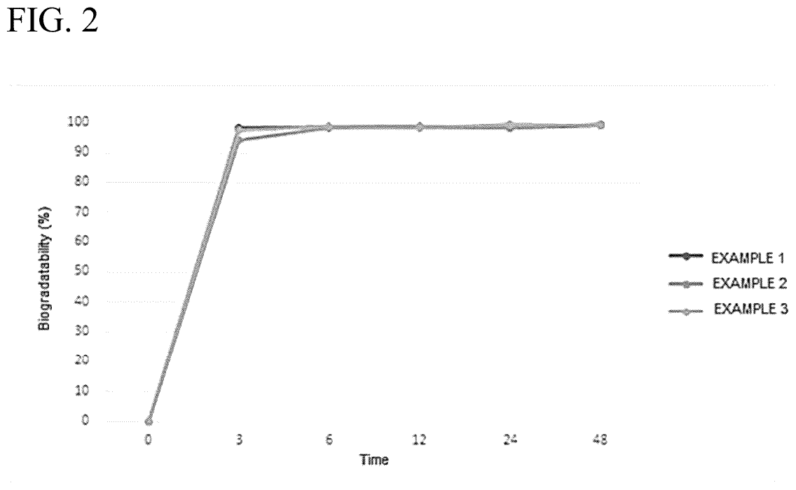

In order to measure the biodegradability of the absorbent bone hemostatic material compositions prepared in Examples 1 to 3 over time, 1 g of the absorbent bone hemostatic material compositions prepared in Examples 1 to 3 was precisely weighted in 20 mL of a phosphate buffer solution (PBS, Ph 7.4) at 37° C., and placed in a conical tube, taken out at a set time in a shaking bath (37° C., 60 RPM), and the amount of the bone hemostatic material biodegraded and reduced was measured as a weight loss rate. The results of calculating the biodegradation according to Equation 1 are shown in Table 1 and FIG. 2 below.

$$[ Equation 1 ]$$

$$Biodegradability = \frac{Initial sample weight - sample weight after 48 hours}{Initial sample weight} \times 100$$

TABLE 1

| | Biodegradability (%) | | |
|---|---|---|---|
| Time (h) | Example 1 | Example 2 | Example 3 |
| 3 | 98.2 | 93.8 | 97.3 |
| 6 | 98.3 | 98.0 | 98.6 |
| 12 | 98.3 | 98.4 | 98.2 |
| 24 | 98.1 | 98.1 | 99.1 |
| 48 | 98.8 | 99.2 | 99.0 |

Referring to Table 1 and FIG. 2, the absorbent bone hemostatic material compositions of Examples 1 to 3 according to the present invention showed a biodegradability of 90% or more after 3 hours and showed a biodegradability of about 98% or more after 6 hours, and it was confirmed that the biodegradability was very excellent. Therefore, even when applied to an actual surgical site, almost all the compositions were biodegraded within 48 hours and it was expected to have a positive effect on bone regeneration.

### Experimental Example 2. Mechanical Property Analysis

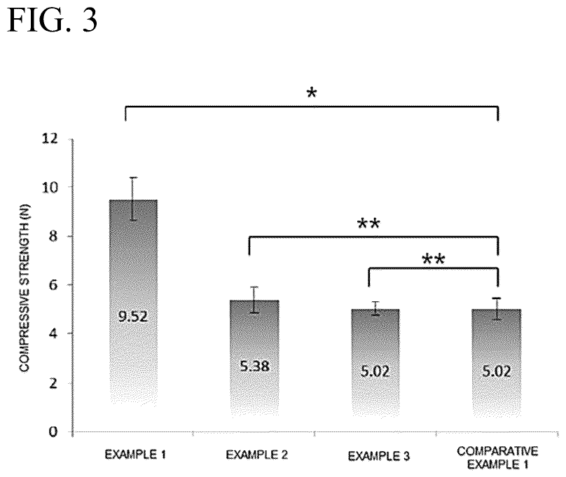

The mechanical properties of the absorbent bone hemostatic material compositions prepared in Examples 1 to 3 and Comparative Example 1 were analyzed. First, analog force gauge (HANDPI NK-10) was used to analyze the compressive strength. Specifically, a sample was placed on a flat plane and a compression force measuring jig of the measuring equipment was mounted at the upper end. Thereafter, the maximum force up to the point at which the sample broke was measured and recorded, and the results are shown in FIG. 3. As illustrated in FIG. 3, it could be confirmed that the hemostatic material composition of Example 1 showed relatively high compressive strength, but the hemostatic material compositions of Examples 2 and 3 and Comparative Example 1 showed almost similar compressive strength.

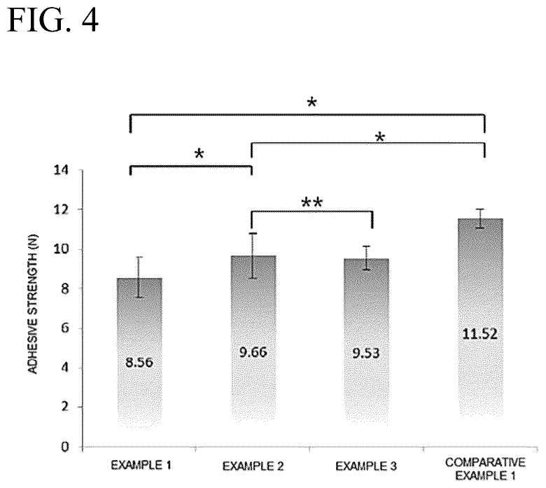

Meanwhile, in order to analyze the adhesive strength, 0.1 g of a sample was kneaded to form a sphere with a diameter of 3 mm and then pressed to a thickness of 1 mm by using the circular jig of the measuring equipment. Thereafter, while lifting the measuring equipment upward, the adhesive strength of the sample was measured and recorded, and the results thereof are shown in FIG. 4. As illustrated in FIG. 4, it could be confirmed that the hemostatic material composition of Example 1 showed relatively low adhesive strength, the hemostatic material composition of Comparative Example 1 showed relatively high adhesive strength, and the hemostatic material compositions of Examples 2 and 3 showed almost similar adhesive strength.

Therefore, it could be confirmed that the hemostatic material compositions of Examples 1 to 3 and Comparative Example 1 did not significantly differ in their physical properties and had almost similar mechanical properties.

### Experimental Example 3. Drug Release Activity Measurement Experiment

The drug release abilities of the absorbent bone hemostatic material compositions prepared in Examples 1 to 3 were measured. Since gentamicin sulfate added as an antibiotic did not have a wavelength detectable by a UV detector, the measurement was performed by using HPLC.

Specifically, after drawing a calibration curve of standard samples 200, 250, 500 and 1,000 ppm, the concentrations of the respective samples were calculated by using a linear regression equation. At this time, the conditions of the test were as follows:
(1) Column: 4.6×150 mm, 5 μm, C18
(2) Mobile Phase: 0.2% TFA: MeOH (98:2)
(3) flow rate: 1 mL/min
(4) Analysis time: 20 min
(5) ELSD Temperature: 60° C.
(6) ELSD gain: 7
(7) Temperature: 30° C.
(8) Injection volume: 10 μL

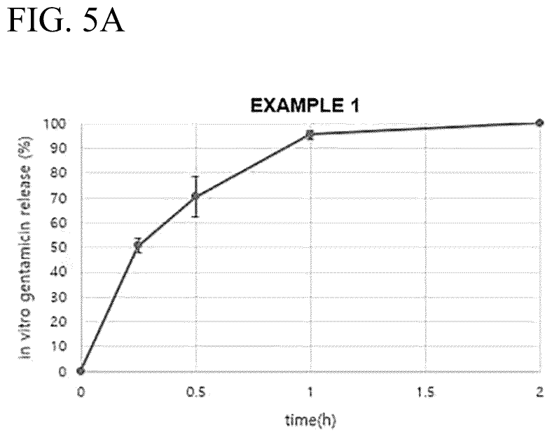

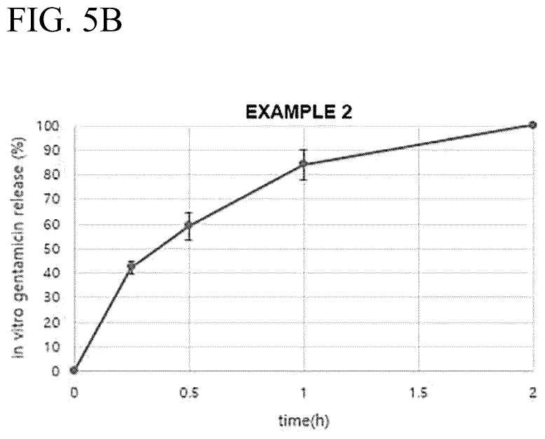

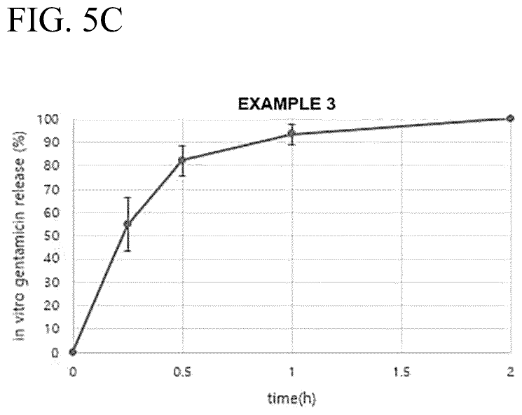

Thereafter, the drug release ability over time was measured and shown in FIGS. 5A to 5C. Referring to FIGS. 5A to 5C, all the absorbent bone hemostatic material compositions prepared in Examples 1 to 3 showed the drug release ability of 100% at 2 hours. This is a result showing a trend similar to the biodegradability measurement experiment. It could be expected that the absorbent bone hemostatic material composition prepared according to the present invention was efficiently applicable to a local site because biodegradation occurred and the drug was immediately released.

### Experimental Example 4. Sterility and Antibacterial Evaluation Test

In order to evaluate the sterility of the absorbent bone hemostatic material compositions prepared in Examples 1 to 3, a test was conducted according to the sterility test method, the eleventh edition of the Korean Pharmacopoeia. The sterility test is a test widely used as a means for evaluating the suitability of sterilization for various medical devices and test substances, and this test evaluated the sterility of the test substance by directly immersing the test substance in the medium.

First, a portion of each of the absorbent bone hemostatic material compositions prepared in Examples 1 to 3 was inoculated into a liquid thioglycolic acid medium and a soybean casein digestion medium. At this time, the liquid thioglycolic acid medium was cultured at 30° C. to 35° C., and the soybean casein digestion medium was cultured at 20° C. to 25° C. On the seventh and fourteenth days after culture, the presence or absence of proliferation of bacteria was observed and shown in Table 2 below.

TABLE 2

| | | Interim | | |
|---|---|---|---|---|
| | | inspection | Last inspection | |
| Medium | Classification | date (7 days) | date (14 days) | Determination |
| Liquid | Test material | N.G. | N.G. | Suitable |
| thioglycolic | Negative | N.G. | N.G. | |
| acid medium | control | | | |
| Soybean | Test material | N.G. | N.G. | Suitable |
| casein | Negative | N.G. | N.G. | |
| digestion | control | | | |
| medium | | | | |

As illustrated in Table 2, as a result of the sterility test of the absorbent bone hemostatic material composition according to the present invention, the proliferation of bacteria could not be confirmed in both the liquid thioglycolic acid medium and the soybean casein digestion medium. Therefore, the absorbent bone hemostatic material composition was determined to be suitable for the eleventh edition of the Korean Pharmacopoeia, the test standard for the sterility test method.

In addition, the antibacterial test was conducted to measure the effect of antibiotic injection. Specifically, Staphylococcus aureus and Escherichia coli were applied to the experiment, and the number of bacteria was measured after 24 hours to measure the antibacterial activity. As a result of the test, both Staphylococcus aureus and Escherichia coli showed an antibacterial activity value of 99.9% or more, and it was confirmed that the antibiotic applied to the absorbent bone hemostatic material composition according to the present invention effectively exhibited its effect.

### Experimental Example 5. In Vivo Efficacy Evaluation

In order to evaluate the in vivo efficacy of the absorbent bone hemostatic material compositions prepared in Example 3 and Comparative Example 1, bone infection was induced in a rat skull defect model by using S. aureus, and each hemostatic material composition was applied thereon. Then, antibiotic effect and safety were evaluated. In addition, the results were compared through the biopsy and the evaluation of biodegradability, hemostasis ability, and inflammatory properties in the blood after transplantation. Meanwhile, groups of rats used in each experiment are summarized and shown in Table 3 below.

TABLE 3
Bacterial count change, inflammation level, histology evaluation
Group 1 (day 3)

| | | | |
|---|---|---|---|
| Group 1-1 | Group 1-2 | Group 1-3 | Group 1-4 |
| (infection X, | (infection O, | (infection X, | (infection O, |
| Example 3) | Example 3) | Comparative | Comparative |
| | | Example 1) | Example 1) |

Group 2 (week 1)

| | | | |
|---|---|---|---|
| Group 2-1 | Group 2-2 | Group 2-3 | Group 2-4 |
| (infection X, | (infection O, | (infection X, | (infection O, |
| Example 3) | Example 3) | Comparative | Comparative |
| | | Example 1) | Example 1) |

Group 3 (week 2)

| | | | |
|---|---|---|---|
| Group 3-1 | Group 3-2 | Group 3-3 | Group 3-4 |
| (infection X, | (infection O, | (infection X, | (infection O, |
| Example 3) | Example 3) | Comparative | Comparative |
| | | Example 1) | Example 1) |

Group 4 (week 4)

| | | | |
|---|---|---|---|
| Group 4-1 | Group 4-2 | Group 4-3 | Group 4-4 |
| (infection X, | (infection O, | (infection X, | (infection O, |
| Example 3) | Example 3) | Comparative | Comparative |
| | | Example 1) | Example 1) |
| 5 rats | 5 rats | 5 rats | 5 rats |

Immediate hemostasis ability, biodegradability evaluation
Group 5

| Group 5-1 (day 3) | Group 5-2 (week 1) |
|---|---|
| 10 rats | 10 rats |

(1) Immediate Hemostasis Ability Evaluation

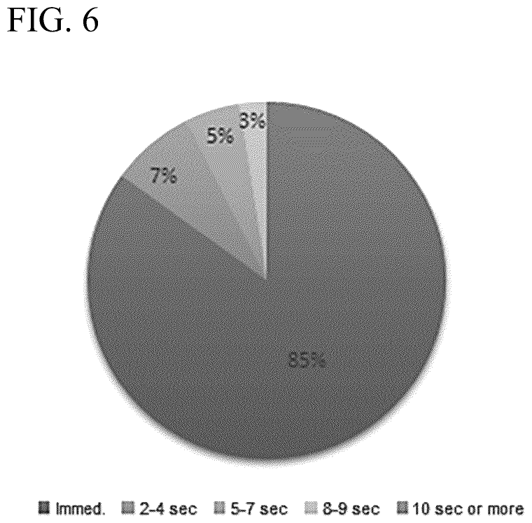

In order to evaluate the immediate hemostasis ability of the absorbent bone hemostatic material compositions prepared in Example 3 and Comparative Example 1, the absorbent bone hemostatic material compositions were applied on each wound, and the time point of hemostasis from 0 seconds to 10 seconds was evaluated and recorded in seconds through video recording. The time point was divided into \textcircled{1} 0-1 second, \textcircled{2} 2-4 seconds, \textcircled{3} 5-7 seconds, \textcircled{4} 8-9 seconds, and \textcircled{5} more than 10 seconds and the evaluation was performed. Meanwhile, the results of the immediate hemostasis evaluation of Example 3 are shown in FIG. 6.

As illustrated in FIG. 6, the absorbent bone hemostatic material composition prepared according to Example 3 of the present invention was evaluated for a total of 40 defects, each two skull defects in 20 subjects, and it was confirmed that the absorbent bone hemostatic material composition showed immediate hemostasis ability of 85%. Meanwhile, it was confirmed that the absorbent bone hemostatic material composition prepared according to Comparative Example 1 showed an immediate hemostasis ability of 73%, and the absorbent bone hemostatic material composition according to the present invention had more excellent immediate hemostasis ability than that of Comparative Example.

(2) Biodegradability Evaluation

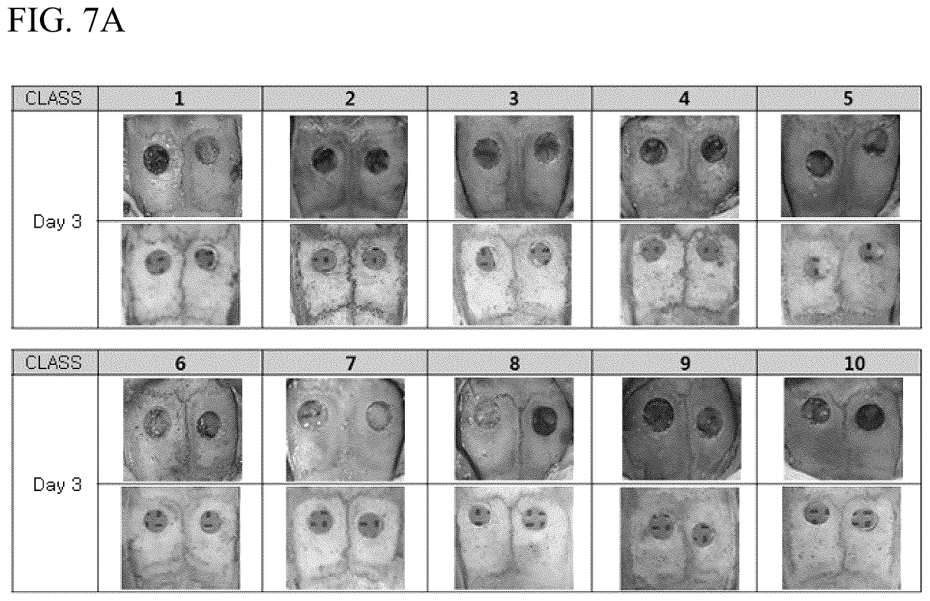

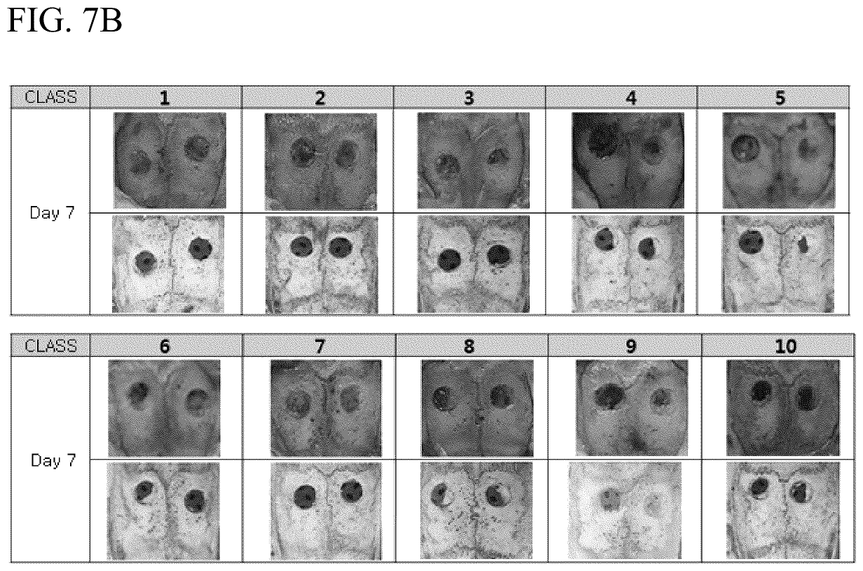

In order to evaluate the biodegradability of the absorbent bone hemostatic material composition prepared in Example 3, autopsies were performed 3 days and 7 days (1 week) after the immediate hemostasis evaluation of the skull of the rat to confirm whether the formulation remained around the wound. The results are shown in FIG. 7A (3 days) and FIG. 7B (7 days). As a result of visual evaluation, it could be confirmed that the formulation was completely biodegraded and disappeared at 3 days and 1 week, and thus, it could be confirmed that the absorbent bone hemostatic material composition according to the present invention had excellent biodegradability.

(3) Antibacterial Evaluation

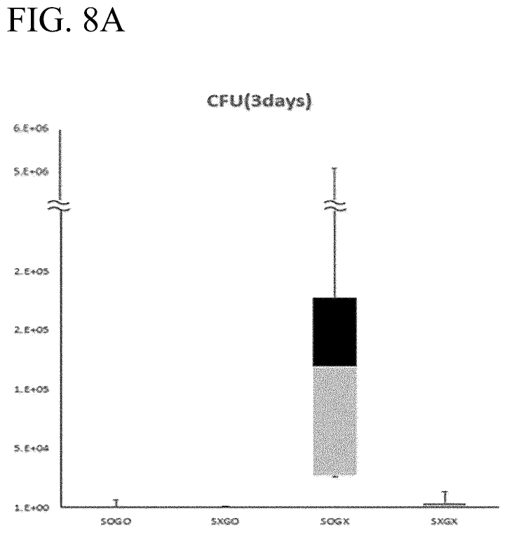

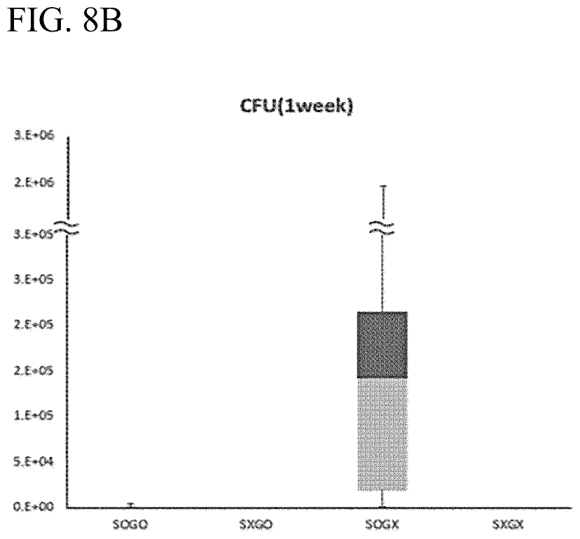

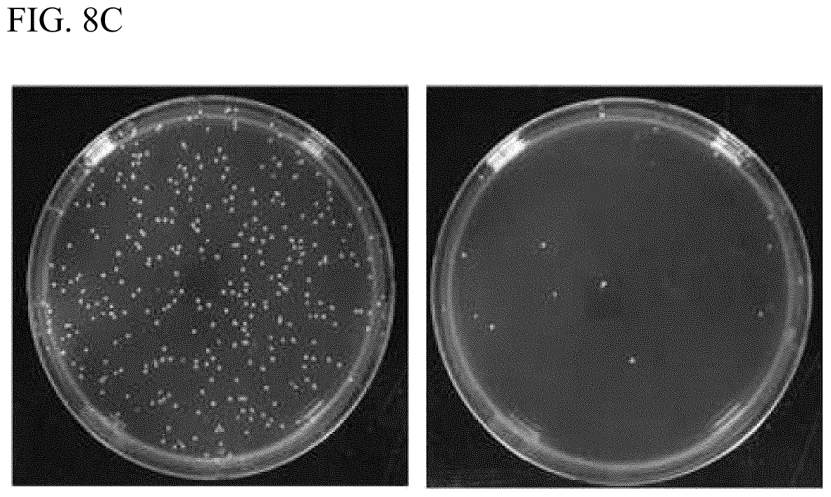

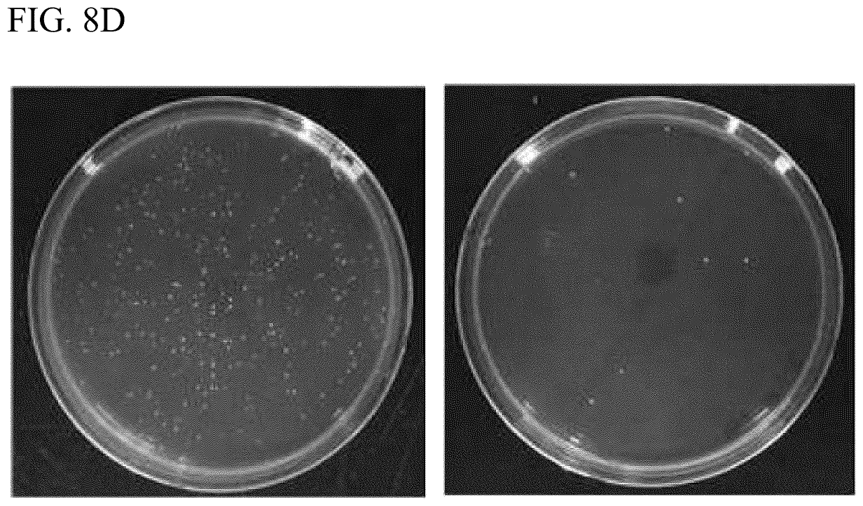

In order to evaluate the antibacterial properties of the absorbent bone hemostatic material compositions prepared in Example 3 and Comparative Example 1, groups were classified into groups 1-1 and 2-1 (SXGO), groups 1-2 and 2-2 (SOGO), groups 1-3 and 2-3 (SXGX), and groups 1-4 and 2-4 (SOGX), and antibacterial activity was evaluated through the presence or absence of bacterial growth in each group. At this time, as shown in Table 3, SO was an S. aureus infected group, SX was an S. aureus non-infected group, GO was a gentamicin-containing group, and GX was a gentamicin-free group. First, after implanting a gelatin sponge on a rat skull, 10⁷ CFU bacteria were dropped on both sides, and the number of bacteria was measured after swabbing the gelatin sponge on both sides with a cotton swab on each observation day. In addition, the results thereof are shown in FIGS. 8A to 8D.

First, FIGS. 8A and 8B are graphs showing the number of bacteria (CFU) for each group after 3 days and 1 week, respectively. As shown in the above graph, it was confirmed that the number of bacteria in the group classified as the SOGX group was significantly higher, and the number of bacteria in the group classified as the SOGO group was almost zero. Meanwhile, the photographs on the left sides of FIGS. 8C and 8D show the bacteria of the SOGX group after 3 days and 1 week, respectively, and the photographs on the right sides of FIGS. 8C and 8D show the bacteria of the SOGO group after 3 days and 1 week, respectively. Even with reference to this, it was confirmed that the number of bacteria in the SOGX medium was significantly higher than the number of bacteria in the SOGO medium.

That is, it could be confirmed that the bone hemostatic material composition (SOGO group) prepared according to Example 3 of the present invention had a significantly superior antibacterial effect, compared to the bone hemostatic material composition (SOGX group) prepared according to Comparative Example 1.

(4) Evaluation of Inflammatory Response and Bone Regeneration Effect Through Tissue Staining

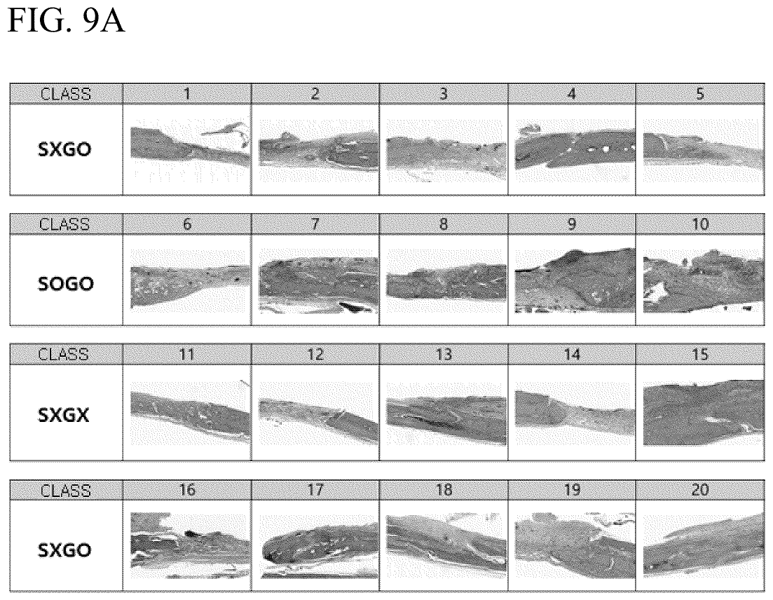

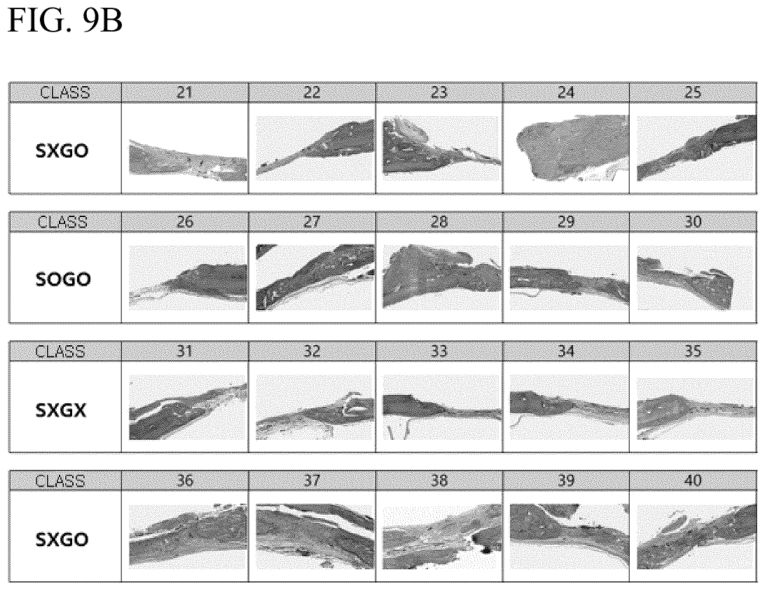

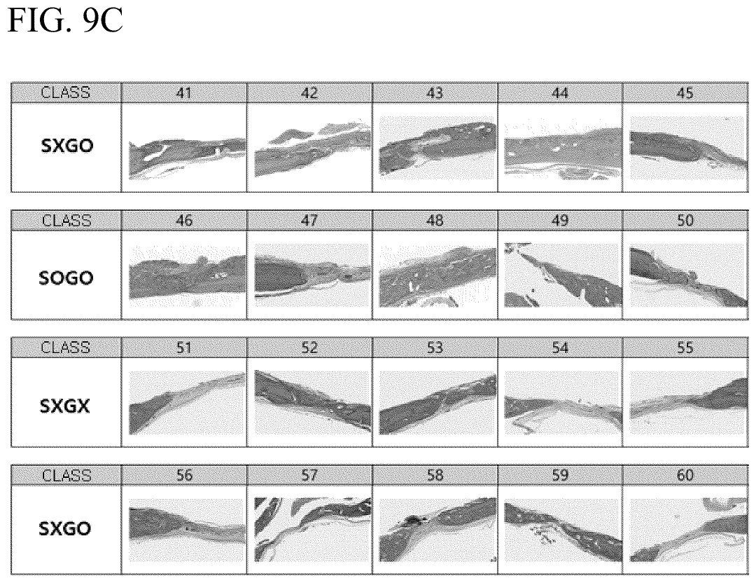

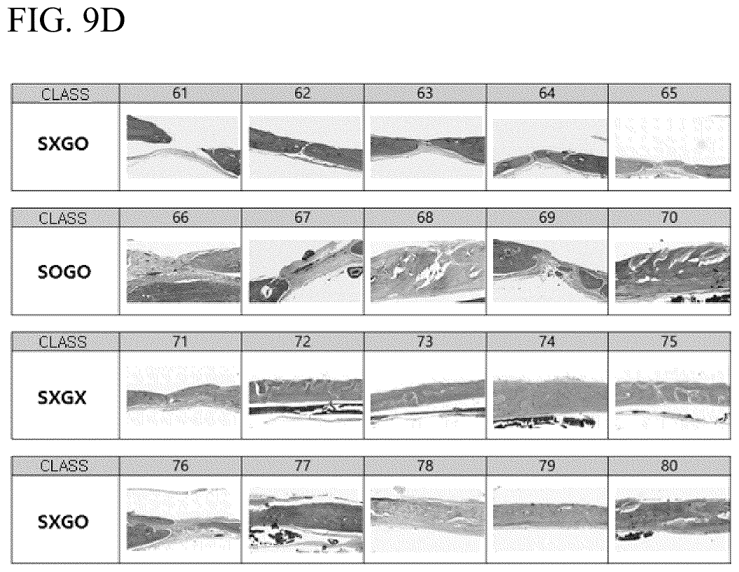

In order to evaluate the inflammatory response and bone regeneration effect of the absorbent bone hemostatic material compositions prepared in Example 3 and Comparative Example 1, the groups were classified into the four groups (SXGO, SOGO, SXGX, and SOGX) as in the section (3) above, and the degree of inflammation and bone formation were confirmed through H&E staining. The results thereof are shown in FIGS. 9A to 9D (FIG. 9A: the third day, FIG. 9B: the first week, FIG. 9C: the second week, FIG. 9D: the fourth week). At this time, as shown in Table 3, SO was an S. aureus infected group, SX was an S. aureus non-infected group, GO was a gentamicin-containing group, and GX was a gentamicin-free group.

Among them, as illustrated in FIG. 9A, it could be confirmed that, in the bone defect part on the third day, the SOGO group had a decrease in inflammatory cells, compared to the SOGX group, and had an increase in the amount and activity of Ostoblast, compared to the other treatment groups.

Accordingly, it could be confirmed that, when applied to the infected bone, the bone hemostatic material composition (SOGO group) prepared according to Example 3 of the present invention showed an excellent effect on inflammation reduction and bone regeneration, compared to the bone hemostatic material composition (SOGX group) prepared according to Comparative Example 1.

### Mode of Disclosure

Hereinafter, the present invention will be described in more detail. However, the present invention may be embodied in various different forms and is not limited by embodiments described herein, and the present invention is only defined by the claims to be described below.

In addition, the terms as used herein are only used to describe specific embodiments, and are not intended to limit the present invention. The singular forms as used herein are intended to include the plural forms as well unless the context clearly indicates otherwise. In the specification of the present invention, the phrase “including a certain element” means “further including other elements” rather than excluding other elements unless otherwise stated.

A first aspect of the present application provides an absorbent bone hemostatic material composition.

The absorbent bone hemostatic material composition includes: a first polymer including a compound represented by Formula 1 or a salt thereof as an active ingredient; a second polymer including a compound represented by Formula 2 or a salt thereof as an active ingredient; and an antibiotic.

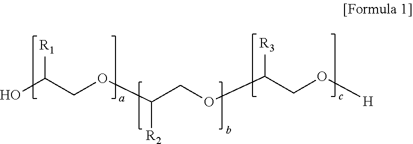

In Formula 1, R1 to R3 may each independently be hydrogen, linear or branched C1-C10 alkyl, linear or branched C1-C10 alkoxy, linear or branched C1-C10 aminoalkyl, linear or branched C2-C10 alkenyl, or linear or branched C2-C10 alkynyl, a and c may each independently be an integer of 2 to 128, and b may be an integer of 16 to 67.

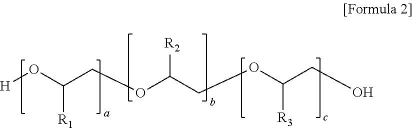

In Formula 2, R1 to R3 may each independently be hydrogen, linear or branched C1-C10 alkyl, linear or branched C1-C10 alkoxy, linear or branched C1-C10 aminoalkyl, linear or branched C2-C10 alkenyl, or linear or branched C2-C10 alkynyl, a and c may each independently be an integer of 2 to 128, and b may be an integer of 16 to 67.

A second aspect of the present application provides an absorbent bone hemostatic material.

The absorbent bone hemostatic material includes the absorbent bone hemostatic material composition according to the first aspect of the present application.

Hereinafter, the absorbent bone hemostatic material composition and the absorbent bone hemostatic material including the same according to the first and second aspects of the present application will be described in detail.

Prior to describing the absorbent bone hemostatic material composition according to the present invention, there has been a problem in that the hemostatic effect of the raw material could not be exhibited 100% in the finished product because the physical properties of the raw material and the physical properties of the finished product were different from each other due to the reaction of each material during mixing while including various materials. In addition, in many cases, a lot of unnecessary materials were included to increase hemostasis ability, and non-compatible materials were included. Therefore, there was also a problem of interfering with bone regeneration. In particular, ‘BONE WAX’ of Ethicon/J&J is applied to a cut surface to provide a physical protective film, but is non-absorbent and remains on the wound continuously. There was a problem of hindering the induction and growth of normal bone cells. Meanwhile, the absorbent bone hemostatic material composition according to the present invention includes a minimum of highly biocompatible bioabsorbent composition, is almost harmless to the human body, and is quickly absorbed and biodegraded. When applied to the bone surface during fractures and bone cutting, the absorbent bone hemostatic material composition provides a physical protective film and has an immediate hemostatic effect. Therefore, the absorbent bone hemostatic material composition may have an effect that does not interfere with normal bone regeneration. In addition, by further including an antibiotic, the absorbent bone hemostatic material composition may have an effect of preventing infection and inflammatory response at the treatment site.

In an embodiment of the present application, in Formulae 1 and 2, R1 to R3 may each independently be hydrogen, linear or branched C1-C5 alkyl, linear or branched C2-C5 alkenyl, or linear or branched C2-C5 alkynyl. Preferably, in Formulae 1 and 2, R1 to R3 may each independently be hydrogen, linear or branched-chain C1-C5 alkyl. More preferably, in Formulae 1 and 2, R1 and R3 may each be hydrogen, and R2 may be linear or branched-chain C1-C3 alkyl. According to an embodiment of the present invention, the first polymer including the compound represented by Formula 1 or a salt thereof as an active ingredient may be poloxamer, and the second polymer including the compound represented by Formula 2 or a salt thereof as an active ingredient may be polyethylene glycol-ran-propylene glycol. Meanwhile, the poloxamer may have a structure in which polyoxypropylene, which is a hydrophobic repeating unit, is included in a central core, and polyoxyethylene, which is a hydrophilic repeating unit, is formed on both sides.

In an embodiment of the present application, the antibiotic is added to prevent infection and inflammatory response at the treatment site, and may include a material selected from the group consisting of, for example, first-generation cephapirin, second-generation ceftazidime, third-generation cefotaxime, and fourth-generation cefepime in cephalosporins, piperacillin in penicillins, gentamicin, amikacin, tobramycin, and neomycin in aminoglycosides, first-generation norfloxacin, second-generation ciprofloxacin, third-generation levofloxacin, and fourth-generation trovafloxacin in quinolones, tetracycline in tetracyclines, ertapenem, meropenem, and imipenem/cilastatin in carbapenems, vancomycin in glycopeptides, complexes thereof, derivatives thereof, and combinations thereof. According to an embodiment of the present invention, the antibiotic may preferably include gentamicin.

In an embodiment of the present application, 50-70 parts by weight of the second polymer and 0.1-20 parts by weight of the antibiotic may be included based on 30-50 parts by weight of the first polymer. Preferably, 1-20 parts by weight of the antibiotic may be included. When the first polymer and the second polymer are included in excess of the above range, the mixing of the composition may not be performed properly. As a result, the hemostatic effect may be inhibited or satisfactory absorption or biodegradation performance may be difficult to obtain. In addition, when the antibiotic is included in an amount below the above range, a relatively low content of the antibiotic may cause infection and inflammatory response at the treatment site. When the antibiotic is included in excess of the above range, the content of the first polymer and the content of the second polymer are relatively low, and thus, biodegradability and hemostatic effect may be inhibited. Meanwhile, the first polymer, the second polymer, and the antibiotic may be properly mixed. When the first polymer, the second polymer, and the antibiotic are properly mixed, it may be meaningful in that the characteristics of the raw material may be maintained.

In an embodiment of the present application, the first polymer may have a melting point of 40° C. to 100° C. When the melting point is lower than 40° C., there is a problem that it is difficult to obtain a hemostatic effect because it is liquefied at body temperature when using a bone hemostatic material. When the temperature is higher than 100° C., the molecular arrangement of the first polymer and the second polymer may not be properly performed because the melting process has to be performed with high heat during the manufacturing process. Therefore, there may be a problem in that it may be difficult to obtain the absorbent bone hemostatic material composition having immediate hemostasis performance and quick absorption and biodegradation performance of the present invention.

In an embodiment of the present application, a weight average molecular weight of the first polymer may be 7,000 to 10,000, and a weight average molecular weight of the second polymer may be 6,000 to 12,000. When the weight average molecular weight of the first polymer is less than 7,000 or greater than 10,000, or when the weight average molecular weight of the second polymer is less than 6,000 or greater than 12,000, the absorbent bone hemostatic material composition including the same may not have an effect of immediate hemostasis and quick absorption and biodegradation. Meanwhile, the weight average molecular weight of the second polymer may be preferably 6,000 to 9,800.

In an embodiment of the present application, the absorbent bone hemostatic material composition may have a hardness of 40 to 80 HS at a temperature of 20° C. to 40° C., and preferably 60 to 80 HS at a temperature of 20° C. to 30° C. and 40 to 60 HS at a temperature of 30° C. to 40° C. When the hardness is less than 40 HS at a temperature of 20° C. to 40° C., it may be too soft and may not properly adhere to the bone, and thus, it may be difficult to obtain satisfactory immediate hemostatic performance. In addition, when the hardness is greater than 80 HS at a temperature of 20° C. to 40° C., it is too hard to form a desired shape, and thus, it may not properly adhere to the bone and it may be difficult to obtain a satisfactory immediate hemostatic effect.

In an embodiment of the present application, the absorbent bone hemostatic material composition may be biodegraded by 90% or more after 3 hours in a phosphate buffer solution (PBS) of pH 7 to 8 at a temperature of 30° C. to 37° C., and preferably 93% or more. In addition, biodegradation may proceed 98% or more after 6 hours under the same conditions, and may proceed about 99% or more after 48 hours. Meanwhile, when biodegradation proceeds for more than 50 hours under the same conditions, an immune response to foreign substances may occur by an immune system, which may interfere with bone formation.

In an embodiment of the present application, the absorbent bone hemostatic material composition may exhibit an immediate hemostatic ability of 0-10 seconds. Specifically, the absorbent bone hemostatic material composition may exhibit excellent immediate hemostatic ability of about 85% immediate hemostasis for 0-1 second, and may complete 100% hemostasis in less than 10 seconds.

In an embodiment of the present application, the absorbent bone hemostatic material composition may have a bacteriostatic reduction rate of 99% or more, preferably about 99.9% or more, after 7 days against Staphylococcus aureus or Escherichia coli. This may be because the absorbent bone hemostatic material composition includes the antibiotic, and due to this, the absorbent bone hemostatic material composition may have a very excellent antibacterial effect.

In an embodiment of the present application, the absorbent bone hemostatic material composition may exhibit a drug release ability of about 80% or more at 1 hour and may exhibit a drug release ability of about 100% at 2 hours. Therefore, the absorbent bone hemostatic material composition may have an excellent effect of efficiently applying the drug to a local site because the drug is immediately released at the same time as biodegradation.

In an embodiment of the present application, the bone mineral density (BMD) after using the absorbent bone hemostatic material composition may be 0.5 to 0.6, and preferably 0.51.

In an embodiment of the present application, the absorbent bone hemostatic material includes the absorbent bone hemostatic material composition and may be characterized in that it is at least one formulation selected from semi-solid formulation and solid formulation. In addition, the absorbent bone hemostatic material may include a groove in the central portion. As described above, since the absorbent bone hemostatic material includes grooves formed in a row in the central portion, the absorbent bone hemostatic material may have an advantage of being easily detached.

A third aspect of the present application provides a method for preparing an absorbent bone hemostatic material composition.

The method for preparing the absorbent bone hemostatic material composition includes: adding, to a reactor, a first polymer including a compound represented by Formula 1 or a salt thereof as an active ingredient, a second polymer including a compound represented by Formula 2 or a salt thereof as an active ingredient, and an antibiotic; heating and stirring the mixture added to the reactor at a temperature of 50° C. to 100° C.; and cooling the mixture.

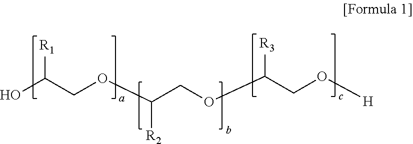

In Formula 1, R1 to R3 may each independently be hydrogen, linear or branched C1-C10 alkyl, linear or branched C1-C10 alkoxy, linear or branched C1-C10 aminoalkyl, linear or branched C2-C10 alkenyl, or linear or branched C2-C10 alkynyl, a and c may each independently be an integer of 2 to 128, and b may be an integer of 16 to 67.

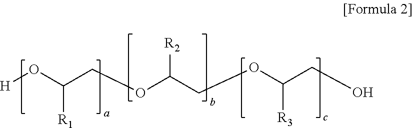

In Formula 2, R1 to R3 may each independently be hydrogen, linear or branched C1-C10 alkyl, linear or branched C1-C10 alkoxy, linear or branched C1-C10 aminoalkyl, linear or branched C2-C10 alkenyl, or linear or branched C2-C10 alkynyl, a and c may each independently be an integer of 2 to 128, and b may be an integer of 16 to 67.

Although detailed descriptions of parts overlapping the first aspect and the second aspect of the present application are omitted, the descriptions of the first aspect and the second aspect of the present application may be equally applied even when the descriptions thereof are omitted in the third aspect.

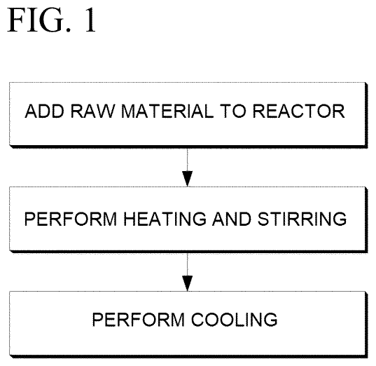

Hereinafter, the method for preparing the absorbent bone hemostatic material composition according to the third aspect of the present application will be described in detail with reference to FIG. 1. At this time, FIG. 1 is a flow chart schematically showing the method for preparing the absorbent bone hemostatic material composition.

First, in an embodiment of the present application, the method for preparing the absorbent bone hemostatic material composition may include adding, to a reactor, a first polymer including a compound represented by Formula 1 or a salt thereof as an active ingredient, a second polymer including a compound represented by Formula 2 or a salt thereof as an active ingredient, and an antibiotic.

In an embodiment of the present application, 50-70 parts by weight of the second polymer and 0.1-20 parts by weight of the antibiotic may be added based on 30-50 parts by weight of the first polymer. Preferably, 1-20 parts by weight of the antibiotic may be added. When the first polymer and the second polymer are added in excess of the above range, the mixing of the composition may not be performed properly. As a result, the hemostatic effect may be inhibited or satisfactory absorption or biodegradation performance may be difficult to obtain. In addition, when the antibiotic is added in an amount below the above range, a relatively low content of the antibiotic may cause infection and inflammatory response at the treatment site. When the antibiotic is added in excess of the above range, the content of the first polymer and the content of the second polymer are relatively low, and thus, biodegradability and hemostatic effect may be inhibited. Meanwhile, the first polymer, the second polymer, and the antibiotic may be properly mixed. When the first polymer, the second polymer, and the antibiotic are properly mixed, it may be meaningful in that the characteristics of the raw material may be maintained.

Next, in an embodiment of the present application, the method for preparing the absorbent bone hemostatic material composition may include heating and stirring the mixture added to the reactor at a temperature of 50° C. to 100° C.

In an embodiment of the present application, the heating temperature may be preferably about 80° C., and each added material may be melted in the above temperature range. When the heating temperature is lower than 50° C., the first polymer, the second polymer, and the antibiotic may not be smoothly stirred. Accordingly, it may be difficult to obtain an absorbent bone hemostatic material composition having an immediate hemostatic effect. In addition, when the heating temperature is higher than 100° C., the molecular arrangement of the first polymer and the second polymer may not be properly formed. Accordingly, it may be difficult to obtain an absorbent bone hemostatic material composition having immediate hemostatic performance and quick absorption and biodegradation performance.

Next, in an embodiment of the present application, the method for preparing the absorbent bone hemostatic material composition may include cooling the mixture.

In an embodiment of the present application, the cooling may be performed at a temperature of −30° C. to 10° C. for 10-60 minutes, and preferably at a temperature of 4° C. for 30 minutes. When the cooling is performed out of the above temperature range, it may be difficult to obtain the absorbent bone hemostatic material composition according to the present invention because the molecules are not properly arranged. In addition, even when the cooling is performed for less than 10 minutes, it may be difficult to obtain the absorbent bone hemostatic material composition having satisfactory effects of immediate hemostasis and quick absorption and biodegradation according to the present invention.

Meanwhile, the cooling may be performed by pouring the mixture into a mold and then adding the mixture to a cooler. In this manner, the solidified absorbent bone hemostatic material composition may be prepared.

The present invention has been described in detail with reference to the preferred embodiments and the drawings, but the scope of the technical idea of the present invention is not limited to these drawings and embodiments. Accordingly, various modifications or equivalents thereof may fall within the scope of the technical idea of the present invention. Therefore, the scope of the technical idea according to the present invention should be interpreted by the claims, and the technical idea within the equivalents should be interpreted as falling within the scope of the present invention.

### INDUSTRIAL APPLICABILITY

As described above, the absorbent bone hemostatic material composition according to the present invention provides an effect of being quickly absorbed and biodegraded in vivo.

The absorbent bone hemostatic material composition according to the present invention provides an immediate hemostatic effect.

The absorbent bone hemostatic material composition according to the present invention has an effect of being easily molded into a desired shape because the hardness of the absorbent bone hemostatic material composition varies with temperature. Accordingly, the absorbent bone hemostatic material composition according to the present invention provides an effect of increasing adhesive force.

The absorbent bone hemostatic material composition according to the present invention includes minimal materials, is harmless to the human body, and mixes properly so that the finished product has the effect of the raw material as it is.

The absorbent bone hemostatic material composition according to the present invention provides the effect of capable of preventing infection and inflammatory response at the surgical site.

1. An absorbent bone hemostatic material composition comprising:
a first polymer including a compound represented by Formula 1 or a salt thereof as an active ingredient;
a second polymer including a compound represented by Formula 2 or a salt thereof as an active ingredient; and
an antibiotic, 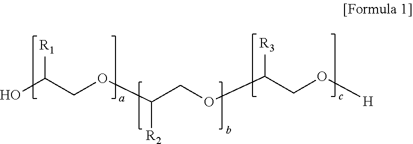
(in Formula 1, R1 to R3 are each independently hydrogen, linear or branched C1-C10 alkyl, linear or branched C1-C10 alkoxy, linear or branched C1-C10 aminoalkyl, linear or branched C2-C10 alkenyl, or linear or branched C2-C10 alkynyl,
a and c are each independently an integer of 2 to 128, and
b is an integer of 16 to 67.) 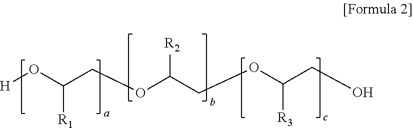
(in Formula 2, R1 to R3 are each independently hydrogen, linear or branched C1-C10 alkyl, linear or branched C1-C10 alkoxy, linear or branched C1-C10 aminoalkyl, linear or branched C2-C10 alkenyl, or linear or branched C2-C10 alkynyl,
a and c are each independently an integer of 2 to 128, and
b is an integer of 16 to 67.)

2. The absorbent bone hemostatic material composition of claim 1, wherein,
in Formulae 1 and 2,
R1 to R3 are each independently hydrogen, linear or branched C1-C5 alkyl, linear or branched C2-C5 alkenyl, or linear or branched C2-C5 alkynyl.

3. The absorbent bone hemostatic material composition of claim 1, wherein,
in Formulae 1 and 2,
R1 to R3 are each independently hydrogen or linear or branched C1-C5 alkyl.

4. The absorbent bone hemostatic material composition of claim 1, wherein,
in Formulae 1 to 2,
R1 and R3 are each hydrogen, and
R2 is linear or branched C1-C3 alkyl.

5. The absorbent bone hemostatic material composition of claim 1, wherein the antibiotic includes a material selected from the group consisting of first-generation cephapirin, second-generation ceftazidime, third-generation cefotaxime, and fourth-generation cefepime in cephalosporins, piperacillin in penicillins, gentamicin, amikacin, tobramycin, and neomycin in aminoglycosides, first-generation norfloxacin, second-generation ciprofloxacin, third-generation levofloxacin, and fourth-generation trovafloxacin in quinolones, tetracycline in tetracyclines, ertapenem, meropenem, and imipenem/cilastatin in carbapenems, vancomycin in glycopeptides, complexes thereof, derivatives thereof, and combinations thereof.

6. The absorbent bone hemostatic material composition of claim 1, wherein 50-70 parts by weight of the second polymer and 0.1-20 parts by weight of the antibiotic are included based on 30-50 parts by weight of the first polymer.

7. The absorbent bone hemostatic material composition of claim 1, wherein a weight average molecular weight of the first polymer is 7,000 to 10,000, and a weight average molecular weight of the second polymer is 6,000 to 12,000.

8. The absorbent bone hemostatic material composition of claim 1, wherein the absorbent bone hemostatic material composition is biodegraded by 90% or more after 3 hours in a phosphate buffer solution (PBS) of pH 7 to 8 at a temperature of 30° C. to 37° C.

9. The absorbent bone hemostatic material composition of claim 1, wherein the absorbent bone hemostatic material composition exhibits an immediate hemostatic ability of 0-10 seconds.

10. The absorbent bone hemostatic material composition of claim 1, wherein the absorbent bone hemostatic material composition has a bacteriostatic reduction rate of 99% or more after 7 days against Staphylococcus aureus or Escherichia coli.

11. An absorbent bone hemostatic material comprising the absorbent bone hemostatic material composition of claim 1.

12. The absorbent bone hemostatic material of claim 11, wherein the absorbent bone hemostatic material is at least one formulation selected from semi-solid formulation and solid formulation.

13. A method for preparing an absorbent bone hemostatic material composition, the method comprising:
adding, to a reactor, a first polymer including a compound represented by Formula 1 or a salt thereof as an active ingredient, a second polymer including a compound represented by Formula 2 or a salt thereof as an active ingredient, and an antibiotic;
heating and stirring a mixture added to the reactor at a temperature of 50° C. to 100° C.; and
cooling the mixture, 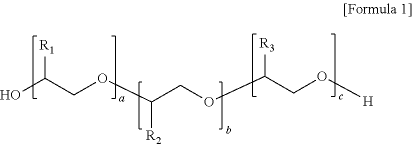
(in Formula 1, R1 to R3 are each independently hydrogen, linear or branched C1-C10 alkyl, linear or branched C1-C10 alkoxy, linear or branched C1-C10 aminoalkyl, linear or branched C2-C10 alkenyl, or linear or branched C2-C10 alkynyl,
a and c are each independently an integer of 2 to 128, and
b is an integer of 16 to 67.) 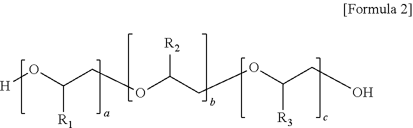
(in Formula 2, R1 to R3 are each independently hydrogen, linear or branched C1-C10 alkyl, linear or branched C1-C10 alkoxy, linear or branched C1-C10 aminoalkyl, linear or branched C2-C10 alkenyl, or linear or branched C2-C10 alkynyl,
a and c are each independently an integer of 2 to 128, and
b is an integer of 16 to 67.)
